(19) United States
(12) Patent Application Publication
(10) Pub. No.: US 2002/0147134 A1
(43) Pub. Date: Oct. 10, 2002

(54) Method of preventing and treating the complications of insulin dependent diabetes mellitus

(76) Inventors: W. Blair Geho, Wooster, OH (US); Hans C. Geho, Solon, OH (US)

Correspondence Address: SDG, INC., HANS C. GEHO, 1350 EUCLID AVENUE, SUITE 200, CLEVELAND, OH, 44115, US

(21) Appl. No.: 09/828,438

(22) Filed: Apr. 6, 2001

(51) Int. Cl. A61K 38/28

(52) U.S. Cl. 514/3

(57) ABSTRACT

This invention relates to a method of preventing, treating, controlling, or delaying the complications of insulin dependent diabetes mellitus by a directed delivery of an insulin to the liver of a host. In particular, the delivery of the insulin is carried out using a polysome or any other Hepatic Directed Delivery (“HDD”) system immediately prior to or during the intake of food by the host.

### BACKGROUND OF THE INVENTION

1. Field of the Invention

This invention relates to a method of preventing and/or treating the complications of insulin dependent diabetes mellitus and, in particular, preventing and treating such complications in a patient suffering therefrom by a directed delivery of an effective amount of an insulin to the liver of the patient.

2. Description of the Related Art

Abnormally low levels of endogenous insulin, caused primarily by a failure of the insulin-producing beta cells of the pancreas, results in the condition known as insulin-dependent diabetes mellitus (“IDDM”).

Insulin is a major anabolic hormone in humans. As such, insulin plays a major role in glucose, lipid, and protein metabolism. One of insulin's roles is to facilitate glucose metabolism by the liver, fat, and muscle tissues of the body. It has been established that in most cases of IDDM, insulin receptor function is normal; it is the supply of insulin itself that is lacking. Specific consequences of this insulin deficiency are hyperglycemia due to unrestrained liver glucose output, elevated levels of free fatty acids, elevated serum ketones, increased levels of triglycerides resulting in increased very low density lipoprotein (“VLDL”) synthesis and decreased VLDL clearance, increased branch-chain amino acids, decreased protein synthesis, and dehydration related to the osmotic effects of elevated blood glucose. These metabolic imbalances are exacerbated by the influence of counterregulatory hormones such as catecholamines and glucagons.

Once beta cell mass is altogether depleted, diabetic patients are completely dependent on exogenous insulin. For diabetics with full-blown insulin dependence, glucose control is highly variable because of the mismatch between insulin requirements and the in vivo availability of exogenous insulin, especially at the liver, fat, and muscle tissues. The present emphasis on very tight control of blood glucose by frequent injections of insulin only emphasizes this mismatch and the inadequacy of exogenous insulin replacement.

A significant and ever-present acute risk for patients with IDDM is ketoacidosis. Insulin deficiency and glucagons enhance the mobilization of free fatty acids supplied by lipolysis, leading to high levels of ketones in the blood. Elevated ketones and hyperglycemia can lead to dehydration and ketoacidosis, which can, in turn, result in death.

Hypoglycemia is more common than diabetic ketoacidosis and is potentially as dangerous as a metabolic emergency (Scientific American Medicine [SAM], 9 Metabolism VI Diabetes Mellitus, page 14; 1997). The risk of hypoglycemia may be lessened by the counterregulatory hormones such as glucagons and norepinephrine which, in the hypoglycemic state, can restore a reasonably normal level of blood glucose. Nevertheless, in many patients with chronic IDDM, this counterregulatory response is lost after about 10 years (Scientific American Medicine [SAM], 9 Metabolism VI Diabetes Mellitus, page 14; 1997). Based on the results of the Diabetes Control and Complications Trial (“DCCT”), clinicians have been urging their patients to attain tight control of their blood glucose levels with more aggressive insulin treatment; however, the cost of aggressive insulin management of this disease is an enhanced risk of potentially life threatening hypoglycemic events.

Long term complications of IDDM are also significant. Most complications are related to resultant microvascular disorders, dyslipidemia, and disorders of fibrinolysis. Clinically, these complications include visual impairment, blindness, renal failure, neuropathies, hypertension, stroke, atherosclerosis and cardiovascular disease. It is also possible that these fundamental disorders may also play a role in abnormal hemostasis and immune function. The cause of these complications is unknown; however, because they occur in forms of diabetes that are genetically distinct but have disordered metabolism in common, some metabolic feature of diabetes most likely causes the complications (Scientific American Medicine [SAM], 9 Metabolism VI Diabetes Mellitus, page 20; 1997). The central role of hyperglycemia being the cause of diabetes-related complications (the glucose hypothesis) is supported by interventional studies in animal models and, convincingly, by the results of the DCCT (Scientific American Medicine [SAM], 9 Metabolism VI Diabetes Mellitus, page 20; 1997).

Risk factors other than hyperglycemia have also been identified. One of the most potent risk factors is the duration of diabetes (Scientific American Medicine [SAM], 9 Metabolism VI Diabetes Mellitus, page 14; 1997).

Clearly with respect to the prevention and treatment of IDDM complications, it is important to address long-term control of hyperglycemia. W. Blair Geho, U.S. Pat. No. 4,603,044 discloses that normalization of glycemic control in IDDM patients requires insulinization of the liver. By delivery of a small amount of insulin to the liver by means of a hepatic targeted vesicle in conjunction with delivery of free, non-hepatic targeted insulin to the periphery (i.e., peripheral muscle and fat [i.e., adipose] cells, normalization of glycemic control can be achieved in animals and humans.

W. Blair Geho, U.S. Pat. No. 4,761,287 additionally provides further insight that normal glucose metabolism requires not only a supply of hepatic insulin but also hepatic serotonin.

A method of preventing, treating, controlling or delaying the onset of the complications resulting from insulin dependent diabetes mellitus is desired.

### SUMMARY OF THE INVENTION

This invention relates to a method of preventing and treating the complications of insulin dependent diabetes mellitus in a host which comprises, delivering by directed means an effective amount of insulin to the liver of the host. Such complications include and are related to chronic hyperglycemia, as well as hypoglycemia, dyslipidemia, microvascular and macrovascular disease including visual impairment and blindness, nephropathy and renal failure, hypertension, stroke, atherosclerosis, cardiovascular disease, neuropathy, and abnormal hemostasis and immune system disorders,.

### DETAILED DESCRIPTION OF THE INVENTION

The present invention relates to a method of preventing, controlling, delaying the onset and treating the complications resulting from insulin dependent diabetes mellitus (not NIDDM). Although it has been previously established that a hepatic dose of insulin delivered at or around mealtime can prevent post-prandial hyperglycemia, it has not been previously established that this same hepatic dose of insulin is required to prevent or control hypoglycemia related to non-hepatic targeted exogenous insulin treatment or to prevent, control, or treat the complications of prolonged hyperglycemia in the insulin-dependent diabetic patient. Current methods of insulin treatment and the prevention and control of diabetes complications have focused instead, as per the DCCT recommendations, on tight peripheral control of hyperglycemia with frequent insulin injections. One of the significant problems with this recommended method of treatment is the heightened risk to the patient of hypoglycemia. In other words, the insulin-dependent diabetic patient seeking to aggressively prevent or manage complications from his or her disease is presently faced with the dangerous risk of hypoglycemia caused by ineffective insulin dosing (i.e., failure of current insulin therapies to involve the liver in glucose metabolism).

It is believed, therefore, that insulin dependent diabetes complications cannot be effectively prevented or treated unless the counterpoint risk of hypoglycemia can be addressed by a method of treatment. It has been surprisingly discovered that insulin, when delivered by a directed administration to the liver of an insulin-dependent diabetic host, is capable of not only preventing post-prandial hypoglycemia, but also preventing the dual complications of fasting hypoglycemia and the more long-range diabetic complications described above (collectively, the complications related to insulin dependent diabetes mellitus will be referred to hereinafter as “complications”).

It is the normalization of hepatic glucose metabolism by the delivery of insulin, in the presence of a normal supply of endogenous serotonin to the liver, that prevents the complications of diabetes mellitus in the purely insulin-dependent diabetic patient. Preventing and treating the various metabolic complications that result from insulin-dependent diabetes mellitus, is achieved by the mealtime or basal directed delivery of an insulin and/or an insulin analogue to their corresponding receptors in the liver. Such delivery restores the normal central role of the liver in managing glucose uptake, storage, and release in the fed and fasted patient.

The present invention provides an insight that has not heretofore been known. The insight is that the primary underlying abnormality in the insulin-dependent diabetic state that results in complications is the lack of a mealtime supply of endogenous insulin to the liver. Current management of insulin-dependent diabetes mellitus by non-hepatic targeted insulin alone, even by frequent insulin injections, including pump injections, is insufficient to prevent these complications, and may actually result in life-threatening hypoglycemia, and thus current therapies for IDDM fail to address this deficiency. Disclosed herein is the expanded significance of normal hepatic glucose metabolism as the central factor in the prevention of IDDM-related complications. Accordingly, not only can IDDM hyperglycemia and hypoglycemia not be adequately and safely prevented without insulinization of the liver, but also that long-term complications of insulin-dependent diabetes mellitus cannot be adequately prevented or treated without directed hepatic insulinization at or near the time a patient eats. In general, hepatic insulinization should be accompanied by insulinization of peripheral fat and muscle tissues as well.

Hepatic insulinization as disclosed herein should first lead to a reduction or elimination of hyperglycemia. In the previous U.S. Pat. Nos. 4,603,044 and 4,863,896, a method of restoring normal hepatic glucose uptake and storage by the delivery of insulin to the liver is described. It is now recognized, however, that this restoration of hepatic glucose uptake and storage is required in order to prevent not only post-prandial hyperglycemia, but the array of complications related to insulin-dependent diabetes mellitus, including hypoglycemia due to exogenous insulin administration.

The term “insulin” shall be interpreted to encompass natural extracted human insulin, recombinantly produced human insulin, insulin extracted from bovine and/or porcine sources, recombinantly produced porcine and bovine insulins, and mixtures of any of these insulin products, and may also encompass fragments or portions of these insulins having insulinergic effects. The term is intended to encompass the polypeptide normally used in the treatment of diabetics in a substantially purified form but encompasses the use of the term in its commercially available pharmaceutical form, which includes additional excipients. The term also includes both short-acting and long-acting insulins as well as insulins with rapid onset of action. The insulin is preferably recombinantly produced and may be dehydrated (dried) or in solution.

The terms “insulin analogue” (or “analog”), “monomeric insulin” and the like are used interchangeably herein and are intended to encompass any form of “insulin” as defined above wherein one or more of the amino acids within the polypeptide chain has been replaced with an alternative amino acid and/or wherein one or more of the amino acids has been deleted or wherein one or more additional amino acids has been added to the polypeptide chain or amino acid sequences which act as insulin in decreasing blood glucose levels. In general, the “insulin analogues” of the present invention include “insulin lispro analogues,” as disclosed in U.S. Pat. No. 5,547,929, incorporated hereinto in its entirety by reference, insulin analogues including LysPro insulin and humalog insulin, and other “super insulin analogues,” wherein the ability of the insulin analogue to affect serum glucose levels is substantially enhanced as compared with conventional insulin as well as hepatoselective insulin analogues which are more active in the liver than in other tissues such as adipose tissue. Preferred analogues are monomeric insulin analogues, which are insulin-like compounds used for the same general purpose as insulin such as insulin lispro, i.e., compounds which are administered to reduce blood glucose levels.

For convenience purposes herein only, whenever the term “insulin” is employed, it shall encompass any agent or component of an “insulin,” as it is defined above.

An effective amount of the insulin is selected. The insulin is then administered to a patient or host, e.g., a human being or other animal, in need thereof for preventing, treating, controlling or delaying the onset of the complications of insulin dependent diabetes mellitus.

The insulin is administered at around the period of time of each mealtime of the host, e.g., a human being or other animal, and delivered to the liver preferably by means of a Hepatic Directed Delivery System (“HDD”), which may be, but is not limited to, a liposome, a polymer, or a combined form of a lipid-polymeric vehicle, to insure a directed delivery to the liver of the host. Additionally, the insulin may be administered as a basal, or fasting, hepatic dose.

A pharmaceutical polysome of the present invention comprises a liposome of a binding agent lipid matrix and a medicament—polymer complex bound to said matrix, where the medicament is insulin. Such a polysome is described in U.S. patent application Ser. No. 09/449,852, filed on Nov. 26, 1999, which is incorporated by reference hereinto in its entirety. Accordingly, the polysome, containing the insulin incorporated therein or thereinto, is made according to the teachings contained in this referenced application.

Alternatively, to also insure directed administration to and hepatocellular uptake of the insulin by the liver, such delivery can be achieved by means of the HDD. The preparation of an HDD system and the incorporation therein of the insulin is also well known. In this regard, reference is made to U.S. Pat. Nos. 4,377,567; 4,603,044; and 5,104,661, all of which are incorporated hereinto by reference in their entirety.

The effective amount of the insulin that is administered will of course be dependent on the subject being treated, the type and severity of the affliction, the manner of administration and the judgment of the prescribing physician. Although effective dose ranges are dependent upon a variety of factors, and are generally known to one of ordinary skill in the art, some dosage guidelines can generally be defined.

Typically, the insulin is present in the bottle (pre-administration) in an amount ranging from about 10 Units/ml to about 1000 Units/ml. Preferably, the effective amount to be delivered to the patient for the prevention, treatment, control, or delay of onset of the complications associated with IDDM is from about 0.01 to about 10 Units of insulin per kilogram of body weight of the patient. As previously described in U.S. Pat. No. 4,863,896 (Combined Forms of Insulin), it may be desirable to administer a simultaneous mixture of hepatic-directed insulin and free (non-hepatic-directed) insulin.

For best results in preventing, treating, controlling or delaying the onset of complications associated with IDDM, the patient in need thereof should have the insulin administered to him or her just prior to or simultaneously with the intake of food, e.g. meals; however, it is also contemplated that this invention may provide similar benefits when administered to enable the patient to achieve a desirable basal level of blood glucose. It is further contemplated that optimal results in terms of preventing, treating, controlling, or delaying the onset of complications associated with IDDM may require a combination of mealtime and basal administrations of the above-described hepatic-directed insulin formulations.

It is to be noted that hepatic-directed insulin may be administered by any conventional means, e.g., orally, subcutaneously, intravenously, by inhalation, as a suppository, etc. In this regard, when the insulin is administered subcutaneously the insulin may be actually released over time from the subcutaneous tissue, thus providing a depot-like basal dose in addition to the short acting-effect.

It should further be noted that there is a class of diabetic patients that have what is commonly known or referred to as “type 2” diabetes. While many of these type 2 patients begin their treatment with oral anti-diabetic medications such as sulfonylureas, a sizeable portion of these patients end up on insulin, either alone or in combination with drugs such as sulfonylureas. Certain of these insulin-dependent type 2 patients may also benefit from hepatic-directed insulin.

1. A method of preventing, treating, controlling or delaying the complications of insulin-dependent diabetes mellitus, including chronic hyperglycemia, as well as hypoglycemia, dyslipidemia, microvascular and macrovascular disease including visual impairment and blindness, nephropathy and renal failure, hypertension, stroke, atherosclerosis, cardiovascular disease, neuropathy, and abnormal hemostasis and immune system disorders which comprises, delivering to a patient, by a directed hepatic delivery, an effective amount of insulin to the liver of said patient immediately prior to, during, or after ingestion or administration of food and/or a liquid containing glucose.

2. The method as defined in claim 1 wherein the insulin is delivered to the liver by means of a polysome or other HDD or other pharmaceutically acceptable carrier.

3. A method of preventing, treating, controlling, or delaying the complications as defined in claims 1 and 2, where said patient is administered a basal supply of hepatic-directed insulin.

4. A method of preventing, treating, controlling, or delaying the complications of insulin-dependent diabetes mellitus as defined in claim 1, wherein a portion of the administered insulin is delivered to the liver by means of a polysome or other HDD or other pharmaceutically acceptable carrier, and another portion of the administered insulin is not hepatic directed but is free to circulate to peripheral fat and muscle tissue.
